(19) United States
(12) Patent Application Publication
(10) Pub. No.: US 2022/0007987 A1
(43) Pub. Date: Jan. 13, 2022

(54) Automated Neural Conduction Velocity Estimation

(71) Applicant: Saluda Medical Pty Ltd, Artarmon (AU)

(72) Inventors: Kai Huang, Artarmon (AU); Matthew Marlon Williams, Artarmon (AU)

(73) Assignee: Saluda Medical Pty Ltd, Artarmon (AU)

(21) Appl. No.: 17/287,501

(22) PCT Filed: Oct. 30, 2019
(86) PCT No.: PCT/AU2019/051197

§ 371 Date: Apr. 21, 2021

(30) Foreign Application Priority Data
Oct. 30, 2018 (AU) 2018904105

(51) Int. Cl. A61B 5/311; A61B 5/294; A61B 5/00; A61N 1/05; A61N 1/36

(52) U.S. Cl. CPC A61B 5/311; A61B 5/294; A61B 5/4836; A61B 5/4848; A61B 2560/0209; A61B 5/7203; A61N 1/0551; A61N 1/36071; A61N 1/36139; A61B 5/686

(57) ABSTRACT

An implantable device, or an associated computer program, for estimating a nerve conduction velocity. A stimulus is applied from one or more stimulus electrodes to a nerve. A digitised neural measurement of at least one compound action potential evoked by the at least one stimulus is obtained from one or more recording electrodes by measurement circuitry. The digitised neural measurement comprises a plurality of data sample points. The digitised neural measurement is processed in order to estimate within subsample precision a temporal position of a feature of interest of the compound action potential. From the estimated temporal position of the feature of interest, and from a propagation distance, a conduction velocity of the compound action potential is determined.

### CROSS-REFERENCE TO RELATED APPLICATIONS

This application claims the benefit of Australian Provisional Patent Application No. 2018904105 filed 30 Oct. 2018, which is incorporated herein by reference.

### TECHNICAL FIELD

The present invention relates to measurement of a neural response to a stimulus, and in particular relates to measurement of a compound action potential by using one or more electrodes implanted proximal to the neural pathway.

### BACKGROUND OF THE INVENTION

There are a range of situations in which it is desirable to apply neural stimuli in order to give rise to a compound action potential (CAP). For example, neuromodulation is used to treat a variety of disorders including chronic pain, Parkinson's disease, and migraine. A neuromodulation system applies an electrical pulse to tissue in order to generate a therapeutic effect. When used to relieve chronic pain, the electrical pulse is applied to the dorsal column (DC) of the spinal cord, referred to as spinal cord stimulation (SCS). Neuromodulation systems typically comprise an implanted electrical pulse generator, and a power source such as a battery that may be rechargeable by transcutaneous inductive transfer. An electrode array is connected to the pulse generator, and is positioned in the dorsal epidural space above the dorsal column. An electrical pulse applied to the dorsal column by an electrode causes the depolarisation of neurons, and generation of propagating action potentials. The fibres being stimulated in this way inhibit the transmission of pain from that segment in the spinal cord to the brain. To sustain the pain relief effects, stimuli are applied substantially continuously, for example at a frequency in the range of 50-100 Hz.

Neuromodulation may also be used to stimulate efferent fibres, for example to induce motor functions. In general, the electrical stimulus generated in a neuromodulation system triggers a neural action potential which then has either an inhibitory or excitatory effect. Inhibitory effects can be used to modulate an undesired process such as the transmission of pain, or to cause a desired effect such as the contraction of a muscle.

The action potentials generated among a large number of depolarising fibres of a single nerve tract sum electrically to form a compound action potential (CAP). The propagation velocity of a CAP is determined largely by the fibre diameter so that for large myelinated fibres as found in the dorsal root entry zone (DREZ) and nearby dorsal column the velocity can be over 60 m·s⁻¹, whereas for small C fibres the conduction velocity can be as little as 1-2 m·s⁻¹. A stimulus regime recruiting mostly large fibres will thus produce a CAP with a higher conduction velocity than a stimulus regime recruiting more small fibres. Conduction velocity is also influenced by other factors including temperature, gender, age and, of particular interest, neuropathies. Such neuropathies include demyelinating conditions such as amyotrophic lateral sclerosis and diabetic neuropathy, and physical trauma such as nerve compression.

Nerve conduction studies are thus performed to investigate the existence or severity of neuropathies. However, these studies typically require that a clinician place skin-mounted stimulus and recording electrode(s) at relatively large distances apart alongside the nerve of interest. Such studies are thus relatively expensive and are typically performed only at a single point in time with many weeks or months between each such study, significantly limiting how much data can be obtained about progression of the condition of interest over time. Nerve conduction velocity is also monitored in some surgical procedures, however once again such monitoring occurs only at that time, and at significant expense.

Further, obtaining an electrical measurement of a compound action potential (CAP) evoked on a neural pathway by an electrical stimulus can be a difficult task, as an observed CAP signal will typically have a maximum amplitude of a few tens of microvolts or less, whereas a stimulus applied to evoke the CAP is typically several volts. Artefact usually results from the stimulus, and manifests as a decaying output of several millivolts or hundreds of microvolts throughout the time that the CAP occurs, presenting a significant obstacle to isolating the much smaller CAP of interest. As the neural response can be contemporaneous with the stimulus and/or the stimulus artefact, CAP measurements present a difficult challenge of implant design. In practice, many non-ideal aspects of a circuit lead to artefact, and as these mostly have a decaying exponential characteristic which can be of either positive or negative polarity, overcoming artefact is difficult.

Evoked responses are less difficult to detect when they appear later in time than the artefact, or when the signal-to-noise ratio is sufficiently high. The artefact is often restricted to a time of 1-2 ms after the stimulus and so, if the neural response is detected after this time window, data can be obtained. This is the case in surgical monitoring where there are large distances between the stimulating and recording electrodes so that the neural response propagation time from the stimulus site to the recording electrodes exceeds 2 ms. However, to characterize responses evoked by a single implant such as responses from the dorsal columns to SCS, for example, high stimulation currents and close proximity between electrodes are required, and therefore the measurement process must overcome contemporaneous artefact directly, greatly exacerbating the difficulty of neural measurement.

Neural implants also operate under tight constraints of battery power and limited processor capacity, complicating efforts to monitor the effect of neurostimulation, whether for a single stimulus or repeatedly over time on an ongoing basis for numerous stimuli.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides an implantable device for estimating a nerve conduction velocity, the device comprising:

at least one stimulus electrode and at least one measurement electrode;

measurement circuitry for obtaining a neural measurement from the or each measurement electrode; and

a processor configured to apply from the or each stimulus electrode to a nerve at least one stimulus, obtain from the measurement circuitry a digitised neural measurement of at least one compound action potential evoked by the at least one stimulus, the digitised neural measurement comprising a plurality of data sample points, the processor further configured to process the digitised neural measurement in order to estimate within subsample precision a temporal position of a feature of interest of the compound action potential, and the processor further configured to determine from the estimated temporal position of the feature of interest and from a propagation distance a conduction velocity of the compound action potential.

According to a second aspect the present invention provides a method for estimating conduction velocity of a neural response, the method comprising:

obtaining digitised measurements of the neural response from at least two electrodes which are at distinct locations along a neural pathway, the digitised measurements comprising a plurality of data sample points;

processing the digitised neural measurement in order to estimate within subsample precision a temporal position of a feature of interest of the compound action potential;

determining from the estimated temporal position of the feature of interest a delay between the time of arrival of the neural response at each respective electrode; and

estimating from the delay, and from knowledge of electrode spacing, a conduction velocity of the neural response.

According to a third aspect the present invention provides a system for determining neural conduction velocity from clinical data of an implanted neuromodulation device, the system comprising:

an implanted neuromodulation device configured to deliver electrical neurostimulation therapy, and configured to capture digitised recordings of neural responses to the electrical neurostimulation therapy, and configured to communicate clinical data including digitised recordings of neural responses to a non-implanted receiver; and

a supervisory device configured to receive clinical data from the implanted neuromodulation device via the receiver, and to execute an automated procedure for determining conduction velocity from the digitised recordings.

According to a fourth aspect the present invention provides a non-transitory computer readable medium for determining conduction velocity from clinical data of an implanted neuromodulation device, comprising instructions which, when executed by one or more processors, causes performance of the following:

receiving clinical data from the implanted neuromodulation device, the clinical data including digitised recordings captured by the implanted neuromodulation device of neural responses to electrical neurostimulation therapy delivered by the implanted neuromodulation device; and

executing an automated procedure for determining conduction velocity from the digitised recordings.

The conduction velocity may be determined using a single measurement at a time. Alternatively, measurements of conduction velocity may be accumulated by applying multiple stimuli and measuring the ECAP from each such stimuli. These measurements could all be obtained from a single electrode. For example, these could be stacked or averaged to improve measurement quality. Alternatively, a plurality of sampled measurements may be processed in order to coarsely locate an ECAP peak (or ECAP feature) temporally in each such measurement, and the coarsely located features from the plurality of measurements may be processed in order to produce an improved temporal location of the feature, such as by finding the average, mean or median temporal location of the coarsely located features. To facilitate temporal separation of the sample points of each measurement from the sample points of the other measurements, a sampling start time may be changed by a sub-sample-period amount between obtaining each such measurement, whether randomly or systematically such as by increments. Such embodiments are particularly advantageous in identifying a temporal location without requiring an assumption that an ECAP amplitude is constant among the respective plurality of measurements, as this is generally not the case.

Alternatively, or preferably additionally, the plurality of measurements may in some embodiments be obtained from different measurement electrodes at different distances from the stimulus site so that a line of best fit can be used to determine conduction velocity from the temporal location of one or more selected ECAP features at each such respective measurement electrode.

In some embodiments of the invention, an implanted device may obtain neural response measurements at a first coarse sampling rate at some times, and may obtain neural response measurements at a second higher sampling rate at other times when it is desired to obtain a conduction velocity measurement.

Embodiments of the third and fourth aspects of the invention may further interpolate between the data sample points a best estimated position of a feature of interest of the compound action potential. In embodiments of the first to fourth aspects of the invention the interpolation may be any suitable non-linear interpolation such as a Gaussian interpolation, a Poisson interpolation, polynomial interpolation, spline interpolation, Rational interpolation using Padé approximant, Trigonometric interpolation using Fourier series, Whittaker-Shannon interpolation or digital filtering techniques such as convolution with a frequency-limited impulse signal. The interpolation may be performed in order to more accurately locate any desired feature of the ECAP, such as the P1 peak, N1 peak, P2 peak, ECAP onset, and/or ECAP zero crossings.

Some embodiments of the invention may further provide for estimating from the conduction velocity of the neural response, or from changes in the conduction velocity of the neural response as detected from one measurement to a next measurement, an approximate proportion of fibre classes recruited to produce the neural response. For example, in such embodiments, a high conduction velocity may be taken as being indicative of recruitment of large fibres such as Aβ fibres. Similarly, an increased conduction velocity from one measurement to a next measurement may be taken as being indicative of increased recruitment of large fibres such as Aβ fibres. Such embodiments may be particularly useful in feedback control of an applied stimulus in order to preferentially recruit Aβ fibres to achieve beneficial therapeutic effects while minimizing recruitment of smaller fibre classes to avoid adverse side effects.

In some embodiments of the invention, a measure of conduction velocity may be obtained both antidromically of the stimulus site and orthodromically of the stimulus site. In such embodiments, the measurements may be used as input to a feedback controller designed to effect differential control of fibre classes recruited in an evoked orthodromic volley as compared to fibre classes recruited in an evoked antidromic volley.

In some embodiments of the invention, a measure of conduction velocity may reveal changes in stimulus efficacy, as for example may arise as a result of implant migration away from the ideal location, postural changes of the patient or changes in tissue properties over time. In such embodiments, the measurement(s) of conduction velocity may be used for feedback control of an applied stimulus in order to adjust device operation for such changes.

Some embodiments of the invention may provide for diagnosing a disease which affects neural conduction velocity, by repeatedly measuring a neural conduction velocity over time, and determining from said measurements whether any changes in neural conduction velocity have occurred which are indicative of the disease. The disease may be diabetes mellitus. Diabetes mellitus reversibly slows the neural conduction velocity in patients having insufficient metabolic control, so that detection of reductions in neural conduction velocity may assist in diagnosing the onset, state or progression of diabetes mellitus, and may for example trigger revision of a treatment schedule.

In some embodiments of the third aspect of the invention, the disease may be central sensitization. Central sensitization tends to reduce the neural conduction velocity, so that detection of reductions in neural conduction velocity may assist in diagnosing the onset, state or progression of central sensitization. In such embodiments, a conduction velocity map of the spinal cord may be obtained by progressively applying stimuli using each of a plurality of electrodes of an implanted electrode array, and using spaced apart measurement electrodes to obtain a measure of the conduction velocity resulting from each stimulus site. Mapping conduction velocity against location then will give an indication of the location(s) at which central sensitization has occurred. Such a map may then be used to optimize the location of therapeutic stimuli, either through a manual fitting of the device or through an automated feedback procedure carried out by an implanted control unit.

References herein to estimation or determination are to be understood as referring to an automated process carried out on data by a processor operating to execute a predefined estimation or determination procedure. The approaches presented herein may be implemented in hardware (e.g., using application specific integrated circuits (ASICS)), or in software (e.g., using instructions tangibly stored on computer-readable media for causing a data processing system to perform the steps described above), or in a combination of hardware and software. The invention can also be embodied as computer-readable code on a computer-readable medium. The computer-readable medium can include any data storage device that can store data which can thereafter be read by a computer system. Examples of the computer readable medium include read-only memory (“ROM”), random-access memory (“RAM”), CD-ROMs, DVDs, magnetic tape, optical data storage device, flash storage devices, or any other suitable storage devices. The computer-readable medium can also be distributed over network coupled computer systems so that the computer readable code is stored and executed in a distributed fashion.

### BRIEF DESCRIPTION OF THE DRAWINGS

An example of the invention will now be described with reference to the accompanying drawings, in which:

FIG. 1 schematically illustrates an implanted spinal cord stimulator;

FIG. 2 is a block diagram of the implanted neurostimulator;

FIG. 3 is a schematic illustrating interaction of the implanted stimulator with a nerve;

FIG. 4 is a simplified illustration of the analog output produced by an amplifier when an ECAP passes a measurement electrode;

FIG. 5 illustrates sampled ECAP measurement data, as output by an ADC;

FIG. 6 illustrates in more detail the conduction velocity measurement process implemented in accordance with one preferred embodiment of the invention;

FIG. 7 illustrates in more detail the conduction velocity measurement process implemented by a clinical data viewer in accordance with an embodiment of the invention;

FIG. 8 illustrates the variability in observed ECAP peak amplitude during a conduction velocity estimation process;

FIG. 9 illustrates single ECAP measurements each obtained in response to a single stimulus;

FIG. 10 is a plot of ECAP peak position;

FIG. 11 illustrates the user interface presented by the clinical data viewer 614 for the purposes of automated conduction velocity estimation

FIG. 12 illustrates ECAP grouping based on the relative location of the measurement electrode to the reference electrode; and

FIG. 13 schematically illustrates a system for feedback controlled neurostimulation in accordance with an embodiment of the invention.

### DESCRIPTION OF THE PREFERRED EMBODIMENTS

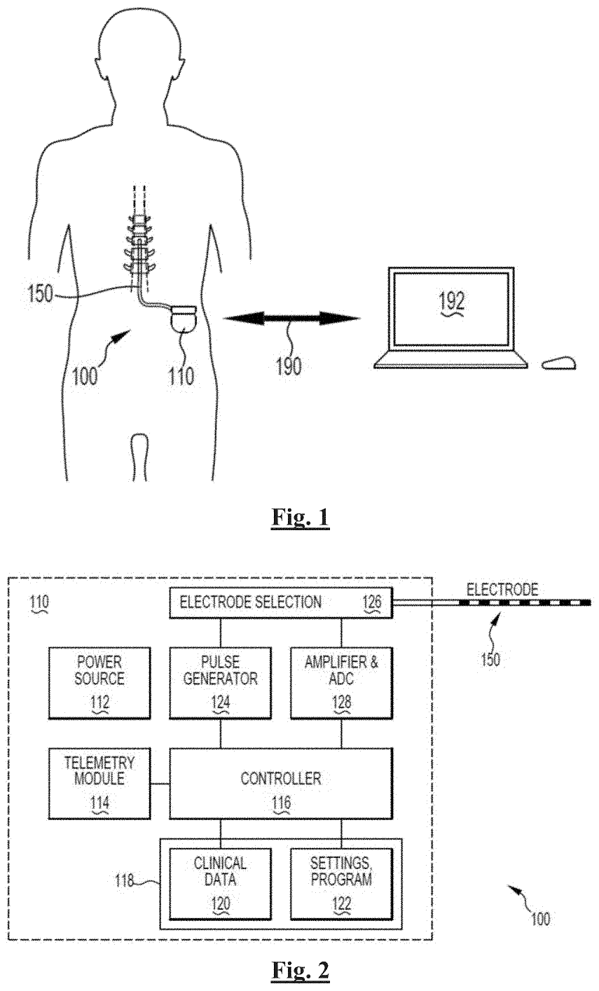

FIG. 1 schematically illustrates an implanted spinal cord stimulator 100. Stimulator 100 comprises an electronics module 110 implanted at a suitable location in the patient's lower abdominal area or posterior superior gluteal region, and an electrode assembly 150 implanted within the epidural space and connected to the module 110 by a suitable lead. Numerous aspects of operation of implanted neural device 100 are reconfigurable by an external control device 192. Moreover, implanted neural device 100 serves a data gathering role, with gathered data being communicated to external device 192 via any suitable transcutaneous communications channel 190.

FIG. 2 is a block diagram of the implanted neurostimulator 100. Module 110 contains a battery 112 and a telemetry module 114. In embodiments of the present invention, any suitable type of transcutaneous communication 190, such as infrared (IR), electromagnetic, capacitive and inductive transfer, may be used by telemetry module 114 to transfer power and/or data between an external device 192 and the electronics module 110. Module controller 116 has an associated memory 118 storing patient settings 120, control programs 122 and the like. Controller 116 controls a pulse generator 124 to generate stimuli in the form of current pulses in accordance with the patient settings and control programs 122. Electrode selection module 126 switches the generated pulses to the appropriate electrode(s) of electrode array 150, for delivery of the current pulse to the tissue surrounding the selected electrode(s). Measurement circuitry 128 is configured to capture measurements of neural responses sensed at sense electrode(s) of the electrode array as selected by electrode selection module 126.

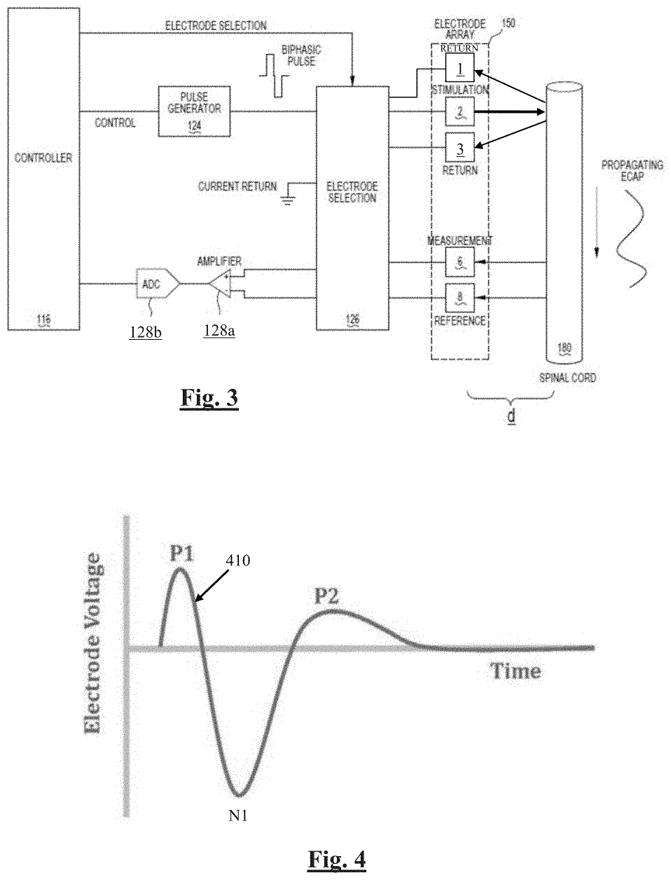

FIG. 3 is a schematic illustrating interaction of the implanted stimulator 100 with a nerve 180, in this case the spinal cord however alternative embodiments may be positioned adjacent any desired neural tissue including a peripheral nerve, visceral nerve, parasympathetic nerve or a brain structure. Electrode selection module 126 selects a stimulation electrode 2 of electrode array 150 to deliver an electrical current pulse to surrounding tissue including nerve 180, and also selects two return electrodes 1 and 3 of the array 150 for stimulus current recovery to maintain a zero net charge transfer. The use of one stimulation electrode 2, and two return electrodes 1 & 3, is referred to herein as tripolar stimulation. FIG. 3 also shows delivery of a biphasic pulse, comprising two temporal phases. However alternative embodiments may deliver stimulation in any suitable form, whether by use of an alternative number of temporal stimulus phases, or by use of an alternative number of stimulus electrodes than that shown in FIG. 3.

Delivery of an appropriate stimulus to the nerve 180 evokes a neural response comprising a compound action potential which will propagate along the nerve 180 as illustrated, for therapeutic purposes which in the case of a spinal cord stimulator for chronic pain might be to create paraesthesia at a desired location. To this end the stimulus electrodes are used to deliver stimuli at any therapeutically suitable frequency, for example 30 Hz, although other frequencies may be used including as high as the kHz range, and/or stimuli may be delivered in a non-periodic manner such as in bursts, or sporadically, as appropriate for the patient. To fit the device, a clinician applies stimuli of various configurations which seek to produce a sensation that is experienced by the user as a paraesthesia. When a stimulus configuration is found which evokes paraesthesia, which is in a location and of a size which is congruent with the area of the user's body affected by pain, the clinician nominates that configuration for ongoing use.

The device 100 is further configured to sense the existence and intensity of compound action potentials (CAPs) propagating along nerve 180, whether such CAPs are evoked by the stimulus from electrodes 1-3, or otherwise evoked. To this end, any electrodes of the array 150 may be selected by the electrode selection module 126 to serve as measurement electrode 6 and measurement reference electrode 8. Signals sensed by the measurement electrodes 6 and 8 are passed to measurement circuitry 128, which for example may operate in accordance with the teachings of International Patent Application Publication No. WO2012155183 by the present applicant, the content of which is incorporated herein by reference. The output of circuitry 128 is digitally sampled by ADC 128 and then used by controller 116 in a feedback arrangement to control the application of subsequent stimuli, and the controller 116 also stores the digitised recording of the neural response, or stores one or more parameters thereof such as ECAP amplitude, to the Clinical Data storage 120.

Device 100, in a typical permanent implantation neuromodulation application such as SCS, will typically operate substantially continuously for many hours of each day, at a stimulus rate in the tens or hundreds of Hz. Being an implanted device powered by battery, the power budget of the device is thus a critical constraint to the functions and design of all aspects of the device operation, including in particular the stimulation regime, the measurement process, and the measurement data analyses and storage. Controller 116 must be a relatively modest computationally efficient processor because battery powered implanted devices cannot support the power load of large processing capability. Data storage 120 is also similarly constrained in size and capability. However, feedback control of stimulus intensity requires that ECAP measurements must be obtained substantially continuously, so that of the order of a million ECAP measurements could be made in the course of a single day. This weighs heavily in favour of reducing the power burden of the measurement process, and one way to do so is to reduce the sampling rate of the ADC 128b.

FIG. 4 is a simplified illustration of the analog output 410 which may be produced by amplifier 128a when an ECAP passes the measurement electrode 6. In practice the output of the amplifier can be heavily influenced by factors such as stimulus artefact, so it is to be borne in mind that the representation in FIG. 4 is simplified. The observed neural response exhibits the typical three-lobed profile, consisting of a positive P1 peak followed by a negative N1 peak and then a second positive P2 peak, as is typical of extracellular recordings of axonal compound action potentials. The first phase P1 is dominated by the capacitive current due to the initial membrane depolarization. P2 is dominated by Na⁺ ion current and is negative due to the influx of Na⁺ ions during the neuronal membrane action potential. The third phase is positive due to the K⁺ ion conduction during repolarization. The duration of the observed response is typically around 2-3 ms and typically has peak amplitudes in the tens of microvolts.

In accordance with the present invention, controller 116 of the device 100 is further configured to measure a conduction velocity of the propagating ECAP upon nerve 180. However, the present invention recognises a number of factors which complicate this task. Device 100 is a single implanted device and so the maximum distance from the stimulus site to a recording electrode is typically no more than about 40-50 mm as constrained by the length of the electrode lead 150. The maximum distance from the stimulus site to a recording electrode is also limited by which electrode is selected as the stimulus electrode in order to optimise the therapy being delivered, so that if the stimulus electrode is near the centre of the lead then the maximum distance from the stimulus site to a recording electrode may be further constrained to be no more than perhaps 20 or 30 mm, and may be as little as about 7 mm. This is important because when calculating conduction velocity by reference to a propagation distance divided by a propagation time, having a small value of distance in the numerator significantly magnifies any errors in the measurement of time in the denominator.

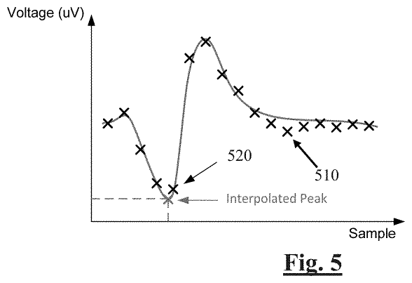

A further complication to the measurement of conduction velocity is that the limited processing and storage capability imposed upon the implanted device mandates that the output 410 of amplifier 128a must be sampled at a relatively slow rate. FIG. 5 illustrates sampled ECAP measurement data, as output by ADC 128b. One such data point is denoted 510. As can be seen, the ADC 128b samples the analog signal relatively coarsely, and relatively few sampled data points are available for subsequent analyses, such as time of arrival analyses for conduction velocity calculations. A direct analysis of only the sampled data points 510 would incorrectly conclude that the N1 peak occurs at sample point 520. This can lead to errors of up to a significant fraction of a millisecond in the value of the measurement of the time of arrival of the ECAP, leading to error in any conduction velocity calculation which is based upon the erroneous time measure. However, the present invention provides for the sample points of the digitised measurement to be processed by a suitable process of interpolation to identify more accurately a position (or time) of the N1 peak, thus significantly improving accuracy of conduction velocity calculations based on such a value while complying with the strict power budget and processor cycles budget which applies to implant 100.

Similar interpolation may additionally or alternatively be performed in order to more accurately locate any desired feature of the ECAP, such as the P1 peak, P2 peak, ECAP onset, and/or ECAP zero crossings.

The present invention thus, in some embodiments, provides a means by which to measure ECAP conduction velocity, using existing implant hardware and software having a sampled ECAP data capturing facility.

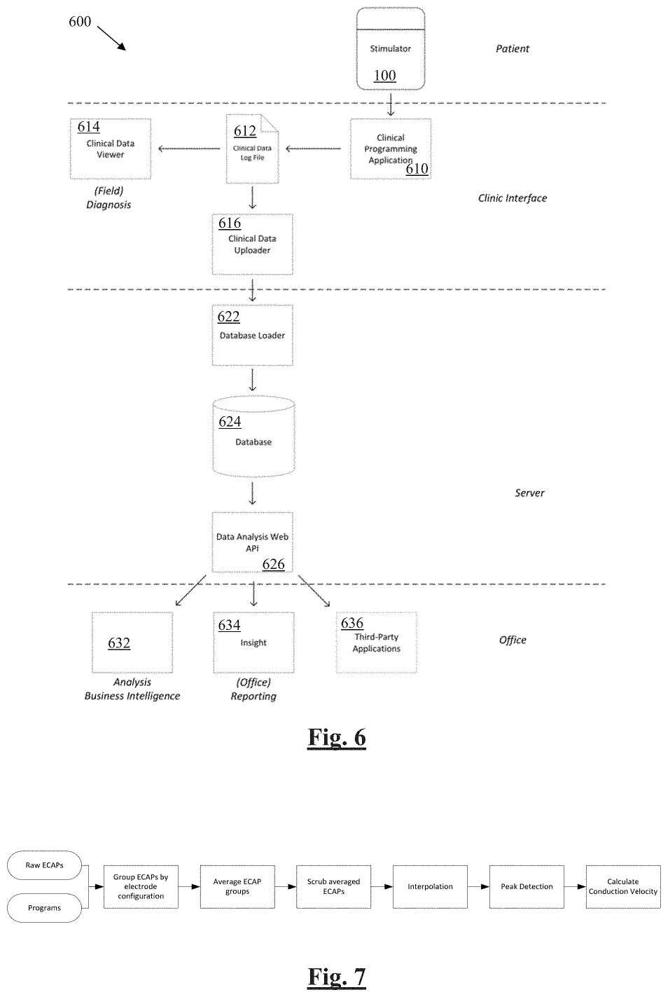

Another embodiment of the invention takes the form of a clinical data viewer software application, configured to retrieve recorded digitised neural measurements obtained by device 100. FIG. 6 illustrates the architecture of a system 600 for managing clinical data of implanted neuromodulation device 100 in accordance with this embodiment of the invention. Stimulator 100 applies stimuli over a potentially long period such as weeks or months and records neural responses, stimulation settings, paraesthesia target level, and other operational parameters, discussed further below. The stimulator 100 comprises a Closed Loop Stimulator (CLS), in that the recorded neural responses are used in a feedback arrangement to control stimulation settings on a continuous or ongoing basis. To effect suitable SCS therapy stimulator 100 may deliver tens, hundreds or even thousands of stimuli per second, for many hours each day. The feedback loop may operate for most or all of this time, by obtaining neural response recordings following every stimulus, or at least obtaining such recordings regularly. Each recording generates a feedback variable such as a measure of the amplitude of the evoked neural response, which in turn results in the feedback loop changing the stimulation parameters for a following stimulus. Stimulator 100 thus produces such data at a rate of tens or hundreds of Hz, or even kHz, and over the course of hours or days this process results in large amounts of clinical data. This is unlike past neuromodulation devices such as SCS devices which lack any ability to record any neural response.

When brought in range with a receiver, stimulator 100 transmits data via telemetry module 114 to a clinical programming application 610 of a clinic interface, which compiles a clinical data log file 612 which is manipulated and optimised and efficiently presented by a clinical data viewer 614, for field diagnosis by a clinician, field clinical engineer (FCE) or the like.

The clinical data viewer 614 is a software application used to analyse data generated by the stimulator 100, and is described in more detail in the present Applicant's International Patent Application No. PCT/AU2018/050278, the content of which is incorporated herein by reference. The clinical data viewer is installed on a Clinical Interface (CI) tablet computer. The clinical data viewer may be installed by trained staff on the CI tablet, or on another computer running any suitable operating system such as Microsoft Windows. Data is collected from the stimulator 100 by the Clinical Programming Application (CPA) 610.

This data is stored by the CPA 610 in a Clinical Data Log (.h5) file 612. The Clinical Data Viewer 614 can open one Clinical Data Log file at a time. The flow of data is shown in FIG. 6. The Clinical Data Uploader 616 is an application that runs in the background on the CI, that automatically uploads files generated by the CPA 610 to a file server. The Data Log Loader 622 is a service which runs on the data server, and monitors the patient data folder for new files and when Clinical Data Log files are uploaded by the Clinical Data Uploader 616 it extracts the data and pushes it to a Database 624.

All logs downloaded from the stimulator 100 are compressed by use of a suitable data compression technique before transmission by telemetry module 114, to enable faster download speeds for logs and/or before storage into the Clinical Data storage 120 so as to allow storage by stimulator 100 of higher resolution data, to provide more data for post-analysis and more detailed data mining for events during use. The server 622, 624, 626 may be configured to provide the user or clinician with a summary of device usage, therapy output, and errors, in a simple single-view page immediately after logs are downloaded upon device connection. The ability to obtain, store, download and analyse such large amounts of neuromodulation data means that the present embodiment can improve patient outcomes in difficult conditions, enable faster, more cost effective and more accurate troubleshooting and patient status, and enable the gathering of statistics across patient populations for later analysis, with a view to diagnosing aetiologies and predicting patient outcomes.

A key component of the system 600 is the Clinical Data Viewer 614, a software application that facilitates the analysis of data stored in Clinical Data Log files 612. The Clinical Data Viewer 614 is intended to be used in the field to diagnose patient issues and optimise therapy for the patient.

FIG. 7 illustrates in more detail the conduction velocity measurement process implemented by the clinical data viewer 614 in accordance with this embodiment of the invention. Digitally sampled ECAPs from a set of known electrode configurations are noise and artefact reduced by per configuration averaging and artefact estimation algorithm respectively. The set of clean ECAPs from the respective configurations are processed by detection algorithm to locate prominent peaks in the sampled time series. Each discrete peak time instance is interpolated to locate a finer resolution peak time instance by functional optimisation using the surrounding ECAP samples. Conduction velocity is estimated as the gradient of the line of best fit of the measuring electrode separation distances with respect to a common reference electrode against the corresponding refined peak time instances.

The embodiment of FIGS. 6 and 7 recognises that manually measuring patient's conduction velocity (CV) under a particular stimulation setting in a clinical setting is an error-prone process, and that it is more efficient for the clinical process to be automated so that the data collection and signal processing are all done under software, taking care of setting the stimulation parameters, stepping through the necessary electrodes, capturing patient data, performing ECAP signal alignment, working out the time offset and finally calculating the conduction velocity based on time offset and known spacing between the measurement electrodes used.

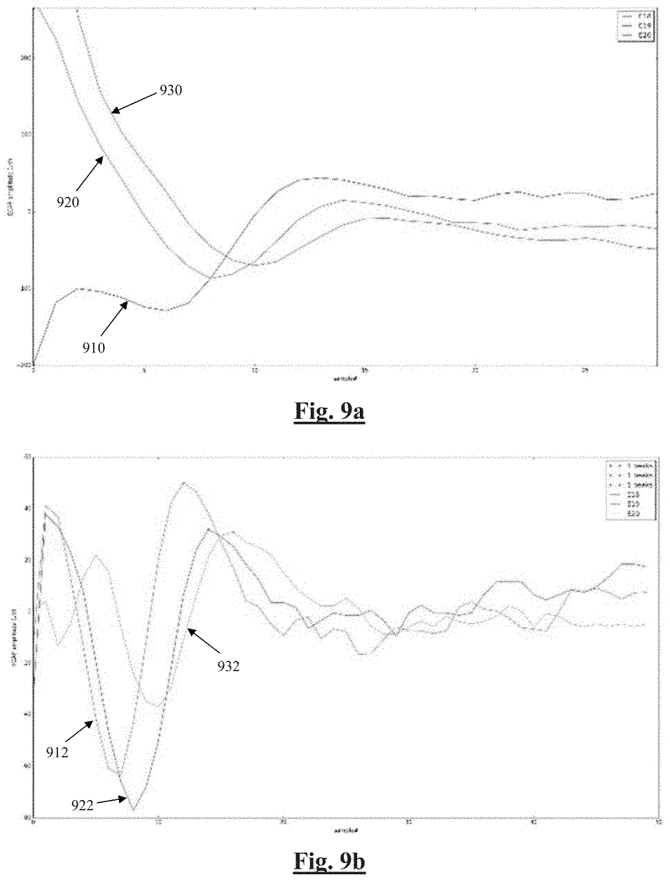

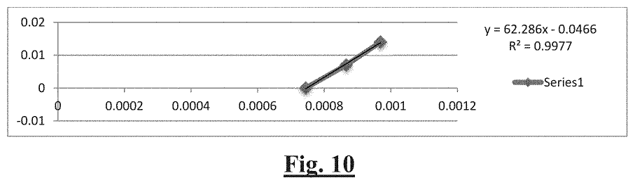

However, difficulties in automating the estimation of conduction velocity include that the ECAP waveforms can be affected by noise, the ECAP waveforms can be affected by drift and polarity inversion of artefacts. Another difficulty is that the ECAP frequencies can be drifting, which in particular complicates the use of template-based matching as set forth for example in the present Applicant's International Patent Publication Nos. WO2015074121 and WO 2017219096 A1, the contents of each being incorporated herein by reference. Further difficulties include that ECAP waveforms can be incomplete, especially for the data from the electrode closest to the stimulation, as is visible in FIG. 9b discussed below.

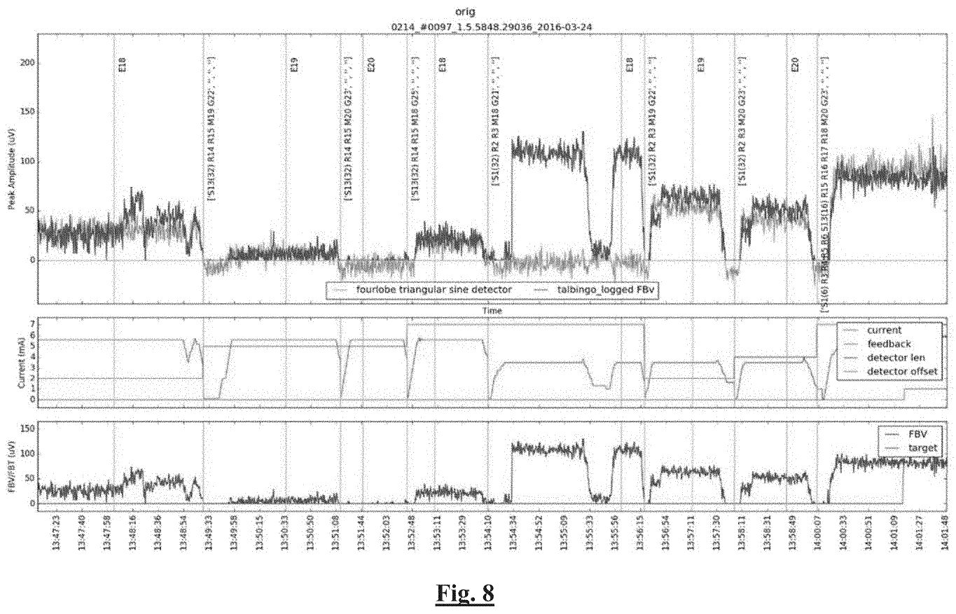

For example, FIG. 8 illustrates the variability in observed ECAP peak amplitude during a conduction velocity estimation process carried out on a human subject over the course of 15 minutes. During this time two sets of manual measurements were taken for the purposes of conduction velocity estimation. This conduction velocity measurement involved obtaining ECAP data from a series of different recording electrodes, of known separations, under the same stimulation setting. Conduction velocity related ECAP recording are labeled by note-entries made by the field clinical engineer, in this case being labeled E18, E19 and E20. As can be seen in FIG. 8 the response to stimulation varied significantly during even this 15 minutes period, illustrating that the manual procedure can be error prone and that multiple recordings need to be collated and cleaned.

The present embodiment recognises that it is more accurate and efficient to configure the CDV 614 to hierarchically arrange the data related to CV measurement. In particular, ECAP samples are arranged in a hierarchical manner in the data logged, for fast retrieval and efficient processing. In this embodiment, these hierarchies consist of level 0, being individual ECAPs (timestamped, with stimulation_current/pulsewidth/polarity/feedback variable attributes). These hierarchies further include level 1, being groups of ECAPs in a common vicinity, with identical stimulation settings from the same recording electrode (neglecting unavoidable variations such as respiration, heart-beat, and slight patient movement). The ECAP sample hierarchies further include level 2, which is a cluster of level-1 recordings from several electrodes in a measurement, and finally level 3 which is long range patient data over days weeks or months, consisting of a variety of electrophysiological measurements.

FIG. 8 illustrates the variation in several ECAP amplitude over many thousands of ECAP measurements in response to many thousands of stimuli. In contrast, FIG. 9 illustrates single ECAP measurements each obtained in response to a single stimulus, and the variation which arises even between single ECAP measurements. FIG. 9A shows digitised sampled ECAP measurements recorded for conduction velocity measurement purposes from three different electrodes, referred to as E18, E19 and E20. In particular, the sampled data from E18 is plotted at 910, the sampled data from E19 is plotted at 920 and the sampled data from E20 is plotted at 930. As can be seen in FIG. 9a, artefact makes it difficult to locate the ECAP peaks. Accordingly, the process of FIG. 7 provides for artefact reduction to be applied to each recording, also referred to as artefact scrubbing. This may for example include double-exponential curve fitting scrubbing, or any suitable artefact scrubbing technique.

After artefact reduction, the scrubbed recordings 912, 922 and 932 take the form shown in FIG. 9b. It can be observed from FIG. 9b that the recordings exhibit peaks at different times, corresponding to the conduction velocity, and thus the described process automatically elicits measurable features from which conduction velocity can be determined, as desired. However, it can also be seen that the digitised sample data is relatively coarsely sampled around each peak, and that the peaks are only separated by about 2 samples, so that peak position determination from these samples alone will lead to significant inaccuracy, of up to around 50% in a worst case, in conduction velocity determination. Accordingly, in the present embodiment the clinical data viewer software application further comprises a module configured to perform Gaussian fitting of a peak based on the integer sample numbers, to yield an interpolated higher resolution peak position. An alternative module could instead apply Poisson fitting, or any other suitable interpolation or estimation method providing subsample precision, for this purpose. The threshold of peak detection is set to 12.5% of the peak positive value in the ECAP data. The peak separation threshold is set to be 50 samples, to detect one prominent peak only. Automatic peak detection is thus effected to a higher resolution.

Another aspect to notice is the reduction rate of ECAP amplitude as the measurement electrode moving further away from the stimulating electrode, from 110 μVpp (E18) to 100 μVpp (E19) to 65 μVpp (E20).

This process then delivers the following results for the data of FIG. 9:

Tsample = 1/16384 6.10352E−05

| distance (mm) | peak location | peak location (s) | distance (m) |
|---|---|---|---|
| 0 | 12.198 | 0.000744507 | 0 |
| 7 | 14.189 | 0.000866028 | 0.007 |
| 14 | 15.872 | 0.00096875 | 0.014 |

Instead of peak position being determined to a single sample, it is interpolated to three decimal places, as shown above. Noting that the sample rate is 16.384 kHz and that the electrode spacing is 7 mm, the peak location in sample position can be converted to a peak location in time, as is also plotted in FIG. 10. The slope of a best fit to this plot is then the conduction velocity, in this case 62.3 m·s⁻¹, as shown in the following table.

| Tₛₐₘₚₗₑ | E_{spacing} | ΔE | ΔN |
|---|---|---|---|
| $\frac{1}{16384} = 61 µs$ | E_{spacing} = 7 mm | 20 − 18 = 2 | 15.872 − 12.198 |

| | |
|---|---|
| CV | 62.3 m/s (the slope from linear regression of the data series) |

This is in contrast to a manual calculation for the sampled data of FIG. 9, which as illustrated in the following table would produce an erroneous conduction velocity estimate of 57.4 m·s⁻¹.

| Tₛₐₘₚₗₑ | E_{spacing} | ΔE | ΔN |
|---|---|---|---|
| $\frac{1}{16384} = 61 µs$ | E_{spacing} = 7 mm | 20 − 18 = 2 | 16 − 12 = 4 |

| | |
|---|---|
| CVref | (7 mm*2)/(61 μs * 4) = 57.4 m/s |

This manual estimation is thus in error by about 8% which is a large error and sufficiently inaccurate to obscure detailed conduction velocity measurement and tracking over time.

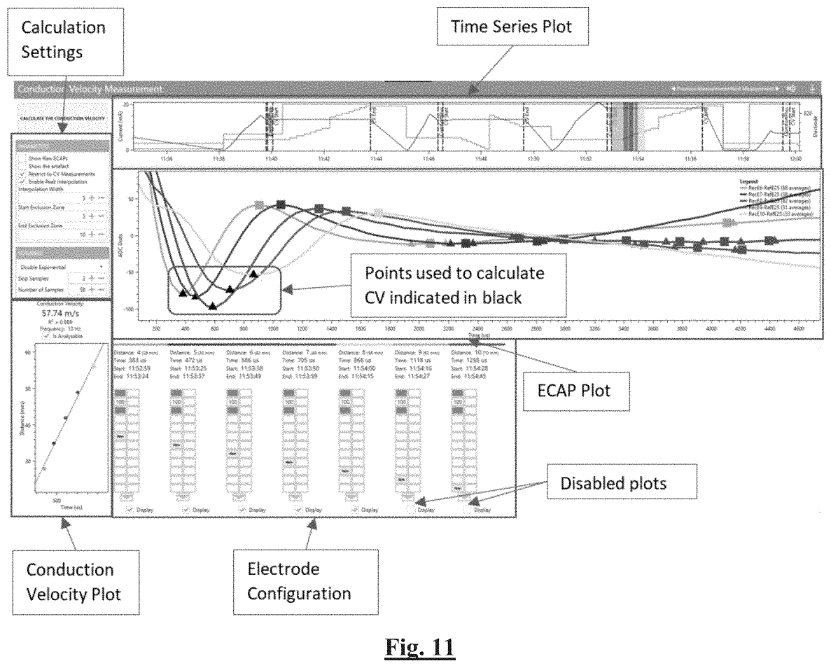

FIG. 11 illustrates the user interface presented by the clinical data viewer 614 for the purposes of automated conduction velocity estimation. The conduction velocity is calculated by pressing the “Calculate the Conduction Velocity” button. This initiates the conduction velocity estimation algorithm which works by averaging ECAPs produced using the same electrode configuration. The peaks of the averaged ECAPs are then detected and plotted on a chart of distance vs. time, denoted the conduction velocity plot in FIG. 11. The slope of the line-of-best-fit through the distance vs. time points is the conduction velocity.

The clinical data viewer 614 allows the following calculation parameters to be changed. Show Raw ECAPs allows the user to see a display of the raw ECAPs used to calculate the average ECAPs. Only averaged ECAPs are shown in FIG. 11. The “Show the artefact” checkbox causes the averaged artefact in the ECAP signal to be displayed. This box is checked by default. The “Restrict to CV Measurements” checkbox restricts measurements to the ‘CV Start’ and ‘CV End’ notes entered during the programming session. The Enable Peak Interpolation checkbox provides for a Gaussian to be fitted through the averaged ECAPs to acquire a more accurate estimation of the ECAP peak. This box is checked by default.

As shown in FIG. 11, the clinical data viewer automatically assesses the averaged ECAPs to identify each local maxima, denoted by squares, and minima, denoted by triangles. The Clinical Data Viewer 614 automatically selects one set of ECAP peaks to use in the conduction velocity calculation. Other peaks can be selected or de-selected by clicking on the square or triangle annotations on the main plot. The peaks that are being used to calculate the conduction velocity are coloured black.

The Time Series plot on the top of FIG. 11 highlights the regions of time corresponding to each electrode configuration that are used to obtain the averaged ECAPs shown in the ECAP Plot. The Time Series plot displays the stimulus current (black trace), recording electrode number (red trace), and reference electrode number (purple trace). The time axis of the plot can be zoomed in to focus on a particular region.

The electrode configuration of each averaged ECAP is displayed below the ECAP Plot at the bottom of FIG. 11. The colour bar above the electrode configuration matches the colour of the averaged ECAP plot. The coloured regions on the Time Series plot also match these colours. To remove an averaged ECAP from the ECAP Plot, the user can un-check the Display button underneath the appropriate electrode configuration.

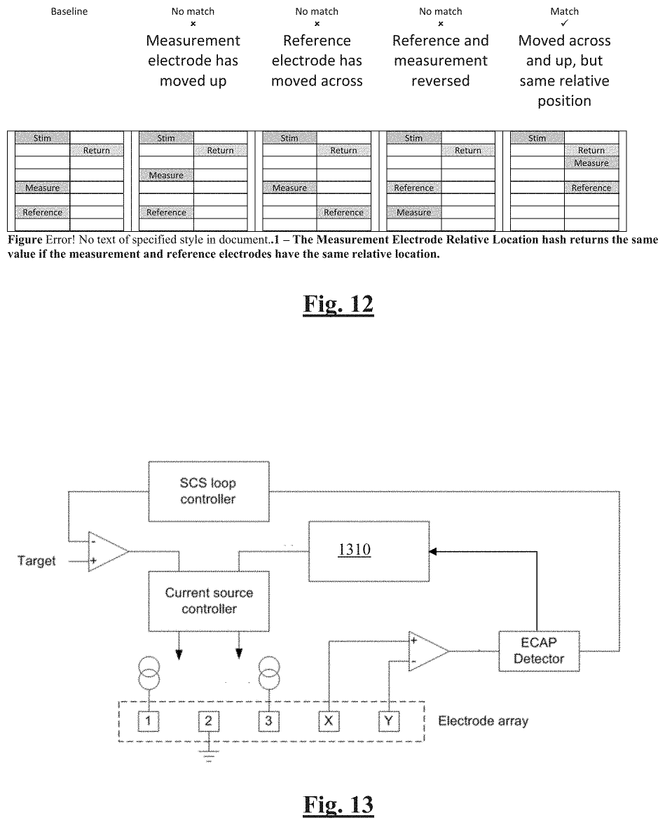

In light of the above, we return to FIG. 7, showing the processing stages undertaken to calculate the conduction velocity. These stages are described in further detail below. In a first stage, Group ECAPs by electrode configuration, the conduction velocity measurement algorithm takes a list of ECAPs and a list of Programs as input. Each ECAP is linked to a program, enabling the electrode configuration to be automatically determined. The ECAPs are grouped based on the electrode configuration using various hashing algorithms. One such algorithm is denoted GetStimulationElectrodeHash, and returns a unique hash code for each combination of stimulation electrodes. Any difference in electrode role, ratio level or ratio max will produce a different output. Stim sets with the same stimulation electrodes but different measurement electrodes will have the same output. Another algorithm is denoted GetMeasurementElectrodeRelativeLocationHash, and returns a unique hash code for the relative location of the measurement electrode to the reference electrode, as illustrated in FIG. 12. Another algorithm is denoted GetOffsetOfStimulationFromMeasurementElectrode, and returns the horizontal offset between the first stimulus electrode and the measurement electrode.

The grouping is done in the order described. ECAPs in a group will have the same hash code for each of the three algorithms.

Other embodiments may also group by stimulation parameters, such as pulse width.

Stage 2 of FIG. 7 is Average ECAP groups. In this stage, for each group, every ECAP is averaged resulting in a single output ECAP. The averaging is intended to reduce the electrical noise in the signal. Stage 3 of FIG. 7 is Scrub average ECAP. In this stage the average ECAP for each group is scrubbed (i.e. the artefact is removed). This allows the peaks to be determined.

Stages 4 and 5 are Interpolation & Peak Detection. Interpolation involves fitting the ECAP samples to a Gaussian, as shown in FIG. 5. Peaks are calculated by finding the local minima or maxima. Using interpolation, the peaks may be anywhere on the fitted curve and are not limited to the sample locations.

Stage 6 is Conduction Velocity Calculation. In this stage the conduction velocity is calculated for each “measurement electrode relative location” group, using the peaks calculated within each “offset of stimulation electrode to measurement electrode” group. For each peak, the distance and time is calculated:

d=E_{spacing}×ΔE

t=Tₛₐₘₚₗₑ×n

where
d is the distance between the stimulation and measurement electrode (mm)
E_{spacing} is the distance between each electrode (7 mm)
ΔE is the number of electrodes between the stimulation and measurement electrodes (vertical offset)
t is the time between stimulation and the ECAP peak being recorded
Tₛₐₘₚₗₑ is the sampling period of the ADC (1/16384 s)
n is the sample index of the peak (n∈ [symbol] without interpolation, n∈ [symbol] with interpolation)
d and t are calculated for each peak and plotted on a curve.

The conduction velocity in mm·s⁻¹ is then the line of best fit through the peaks.

FIG. 13 schematically illustrates a system for feedback controlled neurostimulation in accordance with an embodiment of the invention utilising the above-described principles. In particular, control block 1310 is configured to control ongoing device operation as appropriate in response to detected changes in conduction velocity as may for example indicate recruitment of undesired fibre types. The conduction velocity measurements can thus be used to adjust the stimulation parameters and improve efficiency in a number of ways, including: 1) Maximising recruitment of specific fibre classes by adjustment of stimulus parameters, 2) Differential control of the ascending/descending volleys and associated blocking of these volleys, 3) Automatic variation of stimulation parameters to adjust for changes in use or environment, and 4) Detection of pathology within the underlying stimulated tissue.

A number of basic fibre properties can be determined from the measurement of the compound action potentials. There are a number of neurological conditions and non-neurological conditions which can affect the parameters determined by the conduction velocity measurements and so the measurement techniques of the present invention can serve as a useful diagnostic indicator. The evoked response provides a measure of the properties of the nerve being depolarised. The conduction velocity, fibre diameter and distribution of fibre diameters of the nerve can be determined by measurement of the evoked response.

The method of the present invention may further be used to monitor the effect of a delivered compound, where the compound affects the neural conduction velocity. The administration of compounds (drugs or other chemical therapeutics) to effect a change in the nervous system is common for treatment of a wide number of diseases and disorders. Anaesthetics of various types are administered to the spinal cord for the relief of pain. Perhaps the most common form is administration of anaesthetics in the epidural space for pain relief during child birth. Treatment efficacy may be determined intra-operatively, for example by using a catheter comprising a tube for administration of a drug into the epidural space, and an electrode array. Electrical stimulation can be delivered directly to the spinal cord and the effect of the administered drug can be directly measured in real time.

Alternative embodiments may be suitable for full implantation within the body of a subject and in such embodiments the evoked potential monitoring of the present invention could be used for ongoing administration of an active compound to produce a therapeutic benefit over time. In such embodiments the implanted system could be integrated with an implantable pump to control the administration of the compound.

Any factor which may affect the properties of the compound action potential could be subject to monitoring by the evoked response system described. For instance, the conduction velocity of nerves is slowed reversibly in patients with diabetic mellitus where there is insufficient metabolic control.

The properties of the stimulated neural population may indicate an underlying pathology or the development of an underlying pathology. The detection of the pathology may be used as a control signal for the regulation of the release of a drug. One such pathology is the development of neuropathic pain via central sensitisation, and accordingly some embodiments of the invention may provide for identification of the onset, progression, or state of central sensitization, using an in-situ device in accordance with the present invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. An implantable device for estimating a nerve conduction velocity, the device comprising:
at least one stimulus electrode and at least one measurement electrode;
measurement circuitry for obtaining a neural measurement from the or each measurement electrode; and
a processor configured to apply from the or each stimulus electrode to a nerve at least one stimulus, obtain from the measurement circuitry a digitised neural measurement of at least one compound action potential evoked by the at least one stimulus, the digitised neural measurement comprising a plurality of data sample points, the processor further configured to process the digitised neural measurement in order to estimate within subsample precision a temporal position of a feature of interest of the compound action potential, and the processor further configured to determine from the estimated temporal position of the feature of interest and from a propagation distance a conduction velocity of the compound action potential.

2. The implantable device of claim 1 wherein the processor is further configured to apply a plurality of stimuli and to obtain from the measurement circuitry a plurality of measurements each comprising a digitised neural measurement of the respective ECAP arising from each such stimuli.

3. The implantable device of claim 2 wherein the processor is configured to obtain the plurality of measurements from a single measurement electrode, or from a single measurement electrode pair.

4. The implantable device of claim 2 wherein the processor is configured to process the plurality of measurements in order to coarsely locate a selected ECAP feature temporally in each such measurement, and wherein the processor is further configured to process the coarsely located features from the plurality of measurements in order to produce an improved temporal location of the feature to within subsample precision.

5. The implantable device of claim 4 wherein the processor is configured to process the coarsely located features from the plurality of measurements by finding the temporal mean of the coarsely located features in order to produce the improved temporal location of the feature to within subsample precision.

6. The implantable device of claim 2, wherein the processor is configured to temporally separate the sample points of each measurement from the sample points of the other measurements relative to the respective ECAP, by changing a sampling start time by a sub-sample-period amount between obtaining each such measurement.

7. The implantable device of claim 2 wherein the processor is further configured to obtain at least one digitised neural measurement of at least one compound action potential evoked by the at least one stimulus from a second measurement electrode, the second measurement electrode being at a different distance from the stimulus site.

8. The implantable device of claim 2 wherein the processor is further configured to obtain neural response measurements at a first coarse sampling rate at some times, and to obtain neural response measurements at a second higher sampling rate at other times when it is desired to obtain a conduction velocity measurement.

9. The implantable device of claim 1 wherein the feature of interest of the compound action potential comprises at least one of: a P1 peak, an N1 peak, a P2 peak, an ECAP onset, and an ECAP zero crossing.

10. The implantable device of claim 1 wherein the processor is further configured to estimate from the conduction velocity of the neural response, or from changes in the conduction velocity of the neural response as detected from one measurement to a next measurement, a proportion of fibre classes recruited to produce the neural response.

11. The implantable device of claim 10 wherein the processor is further configured to apply feedback control of an applied stimulus based on the estimated proportion of fibre classes recruited, in order to preferentially recruit selected fibre types to achieve beneficial therapeutic effects.

12. The implantable device of claim 1 wherein the processor is further configured to apply feedback control of an applied stimulus based on the measure of conduction velocity.

13. A method for estimating conduction velocity of a neural response, the method comprising:
obtaining digitized measurements of the neural response from at least two electrodes which are at distinct locations along a neural pathway, the digitised measurements comprising a plurality of data sample points;
processing the digitised neural measurement in order to estimate within subsample precision a temporal position of a feature of interest of the compound action potential;
determining from the estimated temporal position of the feature of interest a delay between the time of arrival of the neural response at each respective electrode; and
estimating from the delay, and from knowledge of electrode spacing, a conduction velocity of the neural response.

14. A system for determining neural conduction velocity from clinical data of an implanted neuromodulation device, the system comprising:
an implanted neuromodulation device configured to deliver electrical neurostimulation therapy, and configured to capture digitised recordings of neural responses to the electrical neurostimulation therapy, and configured to communicate clinical data including digitised recordings of neural responses to a non-implanted receiver; and
a supervisory device configured to receive clinical data from the implanted neuromodulation device via the receiver, and to execute an automated procedure for determining conduction velocity from the digitised recordings.

15. The system of claim 14, wherein the supervisory device is further configured to estimate within subsample precision a temporal position of a feature of interest of the compound action potential.

16. The system of claim 14 wherein the implanted neuromodulation device is further configured to apply a plurality of stimuli and to obtain from the measurement circuitry a plurality of measurements each comprising a digitised neural measurement of the respective ECAP arising from each such stimuli.

17. The system of claim 16 wherein the implanted neuromodulation device is configured to obtain the plurality of measurements from a single measurement electrode, or from a single measurement electrode pair.

18. The system of claim 16 wherein the supervisory device is configured to process the plurality of measurements in order to coarsely locate a selected ECAP feature temporally in each such measurement, and wherein the supervisory device is further configured to process the coarsely located features from the plurality of measurements in order to produce an improved temporal location of the feature to within subsample precision.

19. The system of claim 18 wherein the supervisory device is configured to process the coarsely located features from the plurality of measurements by finding the temporal mean of the coarsely located features in order to produce the improved temporal location of the feature to within subsample precision.

20. The system of claim 16, wherein the supervisory device is configured to temporally separate the sample points of each measurement from the sample points of the other measurements relative to the respective ECAP, by changing a sampling start time by a sub-sample-period amount between obtaining each such measurement.

21. The system of claim 16 wherein the implanted neuromodulation device is further configured to obtain at least one digitised neural measurement of at least one compound action potential evoked by the at least one stimulus from a second measurement electrode, the second measurement electrode being at a different distance from the stimulus site.

22. The system of claim 16 wherein the implanted neuromodulation device is further configured to obtain neural response measurements at a first coarse sampling rate at some times, and to obtain neural response measurements at a second higher sampling rate at other times when it is desired to obtain a conduction velocity measurement.

23. The system of claim 14 wherein the feature of interest of the compound action potential comprises at least one of: a P1 peak, an N1 peak, a P2 peak, an ECAP onset, and an ECAP zero crossing.

24. The system of claim 14 wherein the supervisory device is further configured to estimate from the conduction velocity of the neural response, or from changes in the conduction velocity of the neural response as detected from one measurement to a next measurement, a proportion of fibre classes recruited to produce the neural response.

25. The system of claim 14 wherein the implanted neuromodulation device is further configured to apply feedback control of an applied stimulus based on the measure of conduction velocity.

26. A non-transitory computer readable medium for determining conduction velocity from clinical data of an implanted neuromodulation device, comprising instructions which, when executed by one or more processors, causes performance of the following:
receiving clinical data from the implanted neuromodulation device, the clinical data including digitised recordings captured by the implanted neuromodulation device of neural responses to electrical neurostimulation therapy delivered by the implanted neuromodulation device; and
executing an automated procedure for determining conduction velocity from the digitised recordings.
